(19) United States
(12) Patent Application Publication
(10) Pub. No.: US 2003/0104576 A1
(43) Pub. Date: Jun. 5, 2003

(54) DNA CONSTRUCT, COMPOSITION, FORMULATIONS & METHODS FOR MAKING THE CONSTRUCT & FOR MODULATING EXPRESSION

(76) Inventor: JONATHAN W. NYCE, GREENVILLE, NC (US)

Correspondence Address: VIVIANA AMZEL, PH.D, EPIGENESIS PHARMACEUTICALS, INC., 7 CLARKE DRIVE, CRANBURY, NJ, 08512, US

(21) Appl. No.: 08/319,974

(22) Filed: Oct. 7, 1994

(51) Int. Cl. C12N 15/09

(52) U.S. Cl. 435/69.3

(57) ABSTRACT

Disclosed are attenuated viruses, not naturally occurring, that contain one or more additional methylation sites in the genome of the virus compared to the corresponding wild-type virus. Preferably, the methylation sites are added into the genome of the virus by introducing an additional CG segment into the genome by means of a silent mutation. The attenuated viruses are useful for producing an immune response, including both the production of antibodies in animals for diagnostic use and the induction of protective immunity in a subject. Pharmaceutical formulations and methods of making the attenuated viruses are also disclosed.

This invention was made with Government support under Grant No. RO1 CA47217 from the National Cancer Institute. The Government has certain rights to this invention.

### FIELD OF THE INVENTION

This application concerns singly or multiply attenuated viruses useful as vaccines, where one applied attenuation strategy is to create additional sites for DNA methylation in the viral genome, such additional sites (1) not affecting the amino acid sequence of the virus, and (2) conferring improved host cell control over the expression of the viral genome.

### BACKGROUND OF THE INVENTION

There are at present no vaccines available which are effective against human retroviral infections, and only one which is effective in animals (feline leukemia virus). Various strategies are currently being investigated in attempts to develop effective vaccines against viruses such as the human (HIV) and simian (SIV) immunodeficiency viruses, including subunit vaccines and whole or partial virus vaccines. Clinical trials of potential HIV-1 vaccines have produced almost universal failure; over a dozen large projects, utilizing either peptide vaccines (small fragments of HIV-1 protein, usually the glycoprotein coat) or killed, denatured virus, have failed.

Studies in non-human primates have demonstrated that removal of the nef gene from SIV immunizes monkeys against secondary challenge to SIV. A natural experiment appears to have likewise verified that removal of the nef gene may produce an effective live attenuated vaccine in humans. A population of individuals have been identified who have been infected with HIV for more than one decade, but who show no signs of progressing to AIDS. When the virus infecting these people was isolated and sequenced, it was determined that these particular HIV strains were spontaneous mutations at the nef gene locus; that is, the nef gene had undergone spontaneous deletion. Herein we describe a method to attenuate viruses used in vaccines whose genomes are a target for host cell DNA methylation.

### SUMMARY OF THE INVENTION

The present invention is based on the discovery that DNA methylation sites, in contrast to other dinucleotides, have been preferentially lost during HIV-1 evolution at a rate which far surpasses that of host genes. There is also a loss of methylation sites in DNA viruses and some RNA viruses. DNA methylation is a process by which the five position carbon atom of specific cytosines in DNA are methylated to create 5-methylcytosine. In animal cells, most methylation occurs in the CpG dinucleotide; that is, in cytosines which are immediately 5′ to guanines. Generally, when genes are methylated, they are transcriptionally “silent”—no messenger RNA and hence no protein is produced from them. The present invention employs the active introduction of silent mutations (i.e., that do not affect the amino acid sequence) into the virus genome, such mutations creating new methylation sites not normally present, the methylation of which will impede viral function.

Accordingly, a first aspect of the present invention is an attenuated virus (or “modified virus”), not naturally occurring, containing at least 1 additional methylation site introduced by mutation in the genome of the virus over the corresponding wild-type virus.

A second aspect of the present invention is a DNA encoding a virus as given above (e.g., a cDNA encoding a virus), as well as a vector (e.g., an expression vector) containing the DNA.

A third aspect of the present invention is a pharmaceutical formulation comprising a virus as given above in combination with a pharmaceutically acceptable carrier. The formulation is useful for both raising antibodies in animals, which antibodies specifically bind to the virus and are useful in diagnostic assays and other methods of detecting the virus in both humans and animals; the formulation is useful as a vaccine formulation for producing protective immunity against the virus in an animal or in a human subject.

A fourth aspect of the present invention is a method of producing an immune response (e.g., producing antibodies and/or producing protective immunity) in a subject. The method comprises administering a virus as given above to the subject in an amount effective to produce an immune response in that subject.

A fifth aspect of the present invention is the use of a virus as described above for the preparation of a medicament for producing an immune response in a subject, as described above.

A sixth aspect of the present invention is a method of making an attenuated virus as given above. The method comprises providing a host cell containing an expression vector, the expression vector containing a DNA encoding the attenuated virus, which host cell does not methylate the DNA sufficiently to block the expression of the viral DNA; and expressing the attenuated virus in said host cell. Typically, the host cell is provided in a suitable incubation media, the virus collected from the media after expression therein (with lysis of the host cell, if necessary), and the media either used directly to produce an immune response in a subject, or the virus collected and/or purified from the media and then combined with other ingredients to produce a pharmaceutical formulation.

The foregoing and other objects and aspects of the present invention are explained in detail in the specification set forth below.

### BRIEF DESCRIPTION OF THE DRAWINGS

FIG. 1 illustrates the interruption of the life-cycle of CpG-inserted retrovirus genomes.

FIG. 2 illustrates the CpG content of HIV-1 strain HIVHX2CG (F. Wong-Staal et al., Nature 313, 277-284 (1985).

FIG. 3 illustrates the CpG content of an HIV-1 genome of the present invention, strain HIV-1^{CPG1} (SEQ ID NO: 1).

### DETAILED DESCRIPTION OF THE INVENTION

The nucleotide sequence of an HIV-1 genome (HIV-1^{CPG1}) modified according to the principles described herein is presented by single strand only, in the 5′ to 3′ direction, from left to right. Nucleotides and amino acids are represented herein in the manner recommended by the IUPAC-IUB Biochemical Nomenclature Commission, or (for amino acids) by three letter code, in accordance with 37 CFR §1.822 and established usage. See, e.g., PatentIn User Manual, 99-102 (November 1990)(U.S. Patent and Trademark Office). In nucleotide sequences herein, the internucleotide phosphate linkage is sometimes designated with a “p” positioned between the standard single capital letter for the nucleotide, as in “CpG” for 5′-CG-3′.

1. Viruses

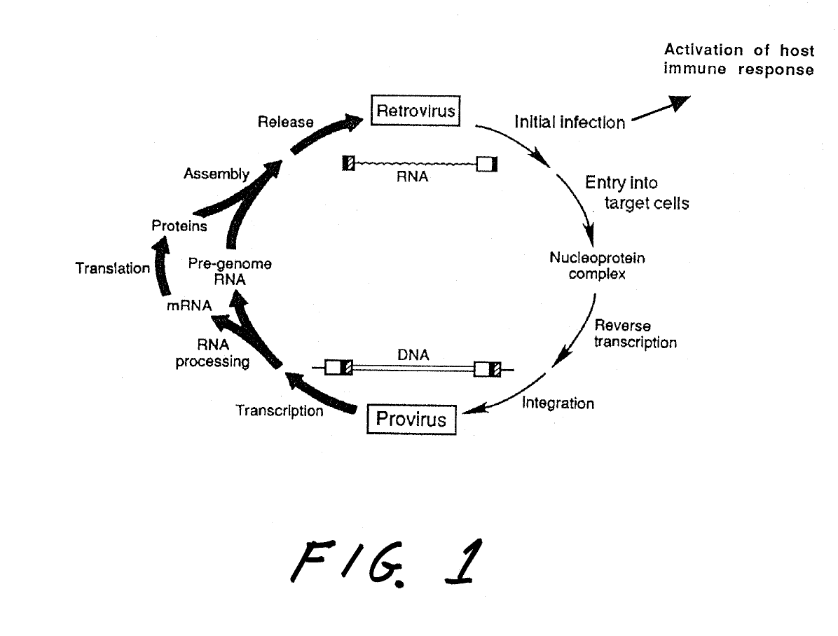

The viruses of the present invention are, in general, expression defective viruses. That is, for the purpose of manufacturing the virus, the virus genome or a DNA encoding the virus genome may be introduced into a host cell that does not methylate the viral DNA sufficient to inactivate it. The viral genome can thus be transcribed into RNA in such a host cell, the RNA then translated into viral proteins, and encapsidated viral genomes (viral particles) produced. For the purpose of producing an immune response in an animal or human subject, the target cells in this case do methylate the viral genome such that methylation sensitive processing of the viral genome, such as transcription, is inhibited therein. The present invention may accordingly be carried out with any virus in which the genome of the virus is methylated in the cells of the subject to which the virus is administered, including DNA viruses, RNA viruses and retroviruses. Retroviruses are particularly preferred. A schematic of the life cycle of a retrovirus and an illustration of how CpG-inserted retrovirus genomes interrupt the life cycle is given in FIG. 1. Note that in FIG. 1, stages of the life cycle depicted by bold lines are interrupted in CpG inserted retrovirions.

Retroviruses that may be used to carry out the present invention include retroviruses of both animals and man. This group of retroviruses includes both simple retroviruses and complex retroviruses. The simple retroviruses include the subgroups of B-type retroviruses, C-type retroviruses and D-type retroviruses. An example of a B-type retrovirus is mouse mammary tumor virus (MMTV). The C-type retroviruses include subgroups C-type group A (including Rous sarcoma virus (RSV), avian leukemia virus (ALV), and avian myeloblastosis virus (AMV)) and C-type group B (including murine leukemia virus (MLV), feline leukemia virus (FeLV), murine sarcoma virus (MSV), gibbon ape leukemia virus (GALV), spleen necrosis virus (SNV), reticuloendotheliosis virus (RV) and simian sarcoma virus (SSV)). The D-type retroviruses include Mason-Pfizer monkey virus (MPMV) and simian retrovirus type 1 (SRV-1). The complex retroviruses include the subgroups of lentiviruses, T-cell leukemia viruses and the foamy viruses. Lentiviruses include HIV-1, but also include HIV-2, SIV, Visna virus, feline immunodeficiency virus (FIV), and equine infectious anemia virus (EIAV). The T-cell leukemia viruses include HTLV-1, HTLV-II, simian T-cell leukemia virus (STLV), and bovine leukemia virus (BLV). The foamy viruses include human foamy virus (HFV), simian foamy virus (SFV) and bovine foamy virus (BFV). The foregoing is illustrative, and is not intended to be limiting of the retroviruses that may be employed in carrying out the instant invention.

Examples of other RNA viruses that may be used in carrying out the present invention include, but are not limited to, the following: members of the family Reoviridae, including the genus Orthoreovirus (multiple serotypes of both mammalian and avian retroviruses), the genus Orbivirus (Bluetongue virus, Eugenangee virus, Kemerovo virus, African horse sickness virus, and Colorado Tick Fever virus), the genus Rotavirus (human rotavirus, Nebraska calf diarrhea virus, murine rotavirus, simian rotavirus, bovine or ovine rotavirus, avian rotavirus); the family Picornaviridae, including the genus Enterovirus (poliovirus, Coxsackie virus A and B, enteric cytopathic human orphan (ECHO) viruses, hepatitis A virus, Simian enteroviruses, Murine encephalomyelitis (ME) viruses, Poliovirus muris, Bovine enteroviruses, Porcine enteroviruses), the genus Cardiovirus (Encephalomyocarditis virus (EMC), Mengovirus), the genus Rhinovirus (Human rhinoviruses—at least 113 subtypes; other rhinoviruses), the genus Apthovirus (Foot and Mouth disease (FMDV); the family Calciviridae, including Vesicular exanthema of swine virus, San Miguel sea lion virus, Feline picornavirus and Norwalk virus; the family Togaviridae, including the genus Alphavirus (Eastern equine encephalitis virus, Semliki forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); the family Rhabdoviridae, including the genus Vesiculovirus (VSV), Chandipura virus, Flanders-Hart Park virus), the genus Lyssavirus (Rabies virus), two genera of fish Rhabdoviruses, and two probable Rhabdoviruses (Marburg virus and Ebola virus); the family Arenaviridae, including Lymphocytic choriomeningitis virus (LCM), Tacaribe virus complex, and Lassa virus; the family Coronoaviridae, including Infectious Bronchitis Virus (IBV), Mouse Hepatitis virus, Human enteric corona virus, and Feline infectious peritonitis (Feline coronavirus).

Illustrative DNA viruses that may be employed in carrying out the present invention include, but are not limited to: the family Poxviridae, including the genus Orthopoxvirus (Variola major, Variola minor, Monkey pox Vaccinia, Cowpox, Buffalopox, Rabbitpox, Ectromelia), the genus Leporipoxvirus (Myxoma, Fibroma), the genus Avipoxvirus (Fowlpox, other avian poxvirus), the genus Capripoxvirus (sheeppox, goatpox), the genus Suipoxvirus (Swinepox), the genus Parapoxvirus (contagious postular dermatitis virus, pseudocowpox, bovine papular stomatitis virus); the family Iridoviridae (African swine fever virus, Frog viruses 2 and 3, Lymphocystis virus of fish; the family Herpesviridae, including the alpha-Herpesviruses (Herpes Simplex Types 1 and 2, Varicella-Zoster, Equine abortion virus, Equine herpes virus 2 and 3, pseudorabies virus, infectious bovine keratoconjunctivitis virus, infectious bovine rhinotracheitis virus, feline rhinotracheitis virus, infections laryngotracheitis virus; the Beta-herpesviruses (Human cytomegalovirus and cytomegaloviruses of swine, monkeys and rodents; the gamma-herpesviruses (Epstein-Barr virus (EBV), Marek's disease virus, Herpes saimiri, Herpesvirus ateles, Herpesvirus sylvilagus, guinea pig herpes virus, Lucke tumor virus; the family Adenoviridae, including the genus Mastadenovirus (Human subgroups A,B,C,D,E and ungrouped; simian adenoviruses (at least 23 serotypes), infectious canine hepatitis, and adenoviruses of cattle, pigs, sheep, frogs and many other species, the genus Aviadenovirus (Avian adenoviruses); and non-cultivatable adenoviruses; the family Papoviridae, including the genus Papillomavirus (Human papilloma viruses, many subtypes, bovine papilloma viruses, Shope rabbit papilloma virus, and various pathogenic papilloma viruses of other species), the genus Polyomavirus (polyomavirus, Simian vacuolating agent (SV-40), Rabbit vacuolating agent (RKV), K virus, BK virus, JC virus, and other primate polyoma viruses such as Lymphotrophic papilloma virus; the family Parvoviridae including the genus Adeno-associated viruses, the genus Parvovirus (Feline panleukopenia virus, bovine parvovirus, canine parvovirus, Aleutian mink disease virus, etc). Finally, DNA viruses may include viruses which do not fit into the above families: Kuru, Creutzfeldt-Jacob disease viruses and chronic infectious neuropathic agents (CHINA virus).

2. Introduction of Attenuating Mutations

The phrase “attenuated virus”, as used herein, means that the infection of a susceptible host by that virus will result in decreased probability of causing i disease in its host (loss of virulence) in accord with standard terminology in the art. See, e.g, B. Davis, R. Dulbecco, H. Eisen, and H. Ginsberg, Microbiology, 132 (3rd ed. 1980). Attenuating mutations are mutations that cause a virus that would otherwise be capable of causing disease to be an attenuated virus. Viruses of the instant invention are attenuated in the sense that the viral life cycle in the susceptible host is inhibited at the level of transcription for retroviruses and DNA viruses. In the case of non-retroviral RNA viruses, the viral life cycle is assumed to be inhibited by loss of function of the RNA genome as a result of CpG methylation.

The number of additional methylation sites introduced by mutation of the genome of a virus as given above to produce a modified virus of the invention may be relatively few (e.g., 1, 2, or 3), or may be at least 10, 50, 100 or 500 or more, depending on the site of the mutation, the nature of the virus, the presence or absence of other attenuating mutations (e.g., a deletion of the nef gene in a retrovirus), etc. Typically, a sufficient number of methylation sites are introduced into the genome of the virus so that the ratio of observed to expected CpG dinucleotides (CpG^{o/e}) within the genome will be increased over that found in the wild type virus 1, 2, 3, 4, 5, 6, 7 or 8-times or more, though the increase in CPG^{o/e} need not be increased as much where a few methylation sites that are particularly active as attenuating mutations are employed.

Modified viruses of the present invention are, in general, infectious virus particles comprising a viral capsid containing the nucleic acid material (DNA or RNA) that comprises the viral genome, which particles bind to the target cells in the subject to which they are administered and introduce their genome into those cells. It is accordingly preferred that the modified virus contain at least two or three mutations that are attenuating (whether by the introduction of a methylation site as described herein or by another mechanism) to reduce the possibility of the virus spontaneously reverting to virulence.

Attenuating CpG mutations of the instant invention are introduced into cDNAs encoding virus by any suitable means, such as by direct synthesis, PCR mutagenesis, or site-directed mutagenesis (see, e.g., U.S. Pat. No. 4,873,192 to Kunkel) (applicant specifically intends that the disclosure of all patent references cited herein be incorporated herein by reference).

The attenuated viruses of the present invention are produced directly on a DNA synthesizing machine, the use of which is known in the art. Specifically, the nucleic acid sequence of the target virus (for example, HIV-1) is selected. The genome is then scanned for non-CpG containing codons which have the possibility of being changed to CpG-containing codons without altering the resulting post-translational amino acid sequence. These non-CpG-containing codons are thus replaced with CpG dinucleotides. For example, a proline coded for by CCT, CCC, or CCA would be switched to CCG. Alternatively, adjacent codons are altered such that they contain a CpG within their adjoining region. As an example, the adjacent codons GCA GTG (alanine-valine) can be altered to GCC GTG, which still codes for alanine-valine but now contains a methylatable CpG (the last C of the first codon and the beginning G of the second).

Of course, certain codons are preferred over others in a species-specific way. It is preferable to create altered genomes by selecting preferred codons where possible (i.e., codons preferred in both the host cell culture system in which the virus is produced, and codons preferred in the subject administered the virus to produce an immune response therein).

Viruses of the present invention can, as noted above, include additional attenuation strategies in addition to the inclusion of the silent CpG mutations described herein. For example, a conventional substitution mutation that produces an amino acid substitution that is attenuating in the encoded protein may also be included, if desired. As another example using HIV-1, the nef gene and another gene or genes or portions thereof can be deleted so as to produce attenuating mutations thereof.

In the case of a retrovirus such as HIV-1, in which many strains of the virus are present, it may be desirable to modify multiple HIV-1 strains by CpG insertion, using them together to produce an effective vaccine.

A novel HIV-1 grime (hereinafter referred to as HIV-1^{CpG-1}) that has been hypersubstituted with noninformational or “silent” CpGs is disclosed hereinbelow. Non-informational means that addition of the CpGs to the genome does not alter the amino acid sequence in the resulting proteins. The hypersubstitution of CpGs makes this novel synthetic genome a target for host cell methylases. Thus, although the virus for which this genome codes is capable of infecting the cell, the proviral genome is easily inactivated by methylation and kept permanently in a dormant state. That is, to the extent the genome can be methylated by the host, it will remain transcriptionally silent.

While the present invention is contemplated primarily for use with so-called “live” virus vaccines, it may also be used with killed virus vaccines, including formaldehyde and heat-inactivated viruses. The instant invention is useful in such vaccine preparations because occasionally live virus escapes the killing procedure and can cause infection. Thus the instant invention, used in conjunction with any other attenuation strategy, provides a further level of attenuation.

3. Production of Virus in Cell Culture

An expression vector is a replicable DNA construct in which a DNA sequence encoding one or more proteins is operably linked to suitable control sequences capable of affecting the expression of the DNA in a suitable host. A replication vector may be used to produce additional DNA where expression of that DNA is not necessary. Choice of host cell for a particular vector will depend upon factors such as whether expression or replication is desired.

Transformed host cells are cells which have been transformed or transfected with vectors constructed using recombinant DNA techniques. Transformed host cells ordinarily express the DNA, but host cells transformed for purposes of cloning or amplifying the target proteins do not need to express the protein.

Suitable host cells generally include prokaryote, yeast or higher eukaryotic cells such as mammalian cells and insect cells. Cells derived from multicellular organisms are a particularly suitable host for recombinant methylated viruses, and insect cells are particularly preferred. Propagation of such cells in cell culture has become a routine procedure (Tissue Culture, Academic Press, Kruse and Patterson, editors (1973)). Examples of useful host cell lines are CD4+T lymphocytes such as MOLT4, VERO and HeLa cells, Chinese hamster ovary (CHO) cell lines, and WI138, BHK, COS-7, CV, and MDCK cell lines. Expression vectors for such cells ordinarily include (if necessary) an origin of replication, a promoter located upstream from the DNA encoding the methylatable virus to be expressed and operatively associated therewith, along with a ribosome binding site, an RNA splice site (if intron-containing genomic DNA is used), a polyadenylation site, and a transcriptional termination sequence.

Where the host cell contains a methylation system that would otherwise methylate the viral genome, that methylation system must be inactivated sufficiently to permit production of the virus therein. Such inactivation may be accomplished by any suitable means, -such as by including a demethylating agent or methylase inhibitor such as 5-azacytidine or 5-azadeoxycytidine in the cell culture media in an amount sufficient to inhibit the methylation system (e.g., 1-10 μM), by adding an antisense oligonucleotide to the media in an amount effective to inactivate the methylation system, or by genetically engineering the cells to express an antisense agent therein effective to inactivate the methylation system. Where the antisense system is genetically engineered into the cell, it is most preferable to use an inducible expression vector, for example on in which the antisense oligonucleotide is placed downstream of a promoter such as the mouse metallothionein promoter, which can be activated to express the antisense by addition of a metal (such as cadmium) to the tissue culture medium. Numerous such inducible expression systems are known to those skilled in the art.

Expressing live virus is particularly feasible in a Baculovirus expression system, which utilizes insect cells as the host cells and viral vectors indigenous to insects (See generally U.S. Pat. Nos. 4,745,051 and 4,879,236 to Smith et al.). Baculoviruses are members of the family Baculoviridae and the genus Baculovirus. The genus comprises three subgroups of viruses: the nuclear polyhedrosis viruses (NPV), the granulosis viruses (GV) and the non-occluded viruses. NPVs include Autographica californica NPV (AcNPV), Heliothis zea NPV (HZNPV) and Bombyx mori NPV (BmNPV). The use of recombinant baculovirus vectors to express foreign proteins in insect cell cultures or larvae is known. See e.g., Luckow & Summers, Bio/Technology, 6, 47 (1988); Tomalski & Miller, Nature, 352, 82 (1991). The use of baculoviruses in this invention is particularly useful because insect host cells (e.g., cultured Spodoptera frugiperda cells) do not possess DNA methylase enzymes and cannot therefore transcriptionally inactivate the viral proviral DNA. In general, a baculovirus expression vector comprises a baculovirus genome containing the DNA to be expressed inserted into the polyhedrin gene at a position where it is under the transcriptional control of the baculovirus polyhedrin promoter.

Modified virus produced by tissue culture techniques as described above can be isolated and/or purified as desired by techniques such as ultrafiltration, and then combined with other ingredients to provide the modified virus in a pharmaceutically acceptable carrier.

4. Pharmaceutical Formulations

A composition of matter comprising an preparation of the attenuated viral particles produced by the cell line of the present invention is disclosed herein. This composition may include any pharmaceutically acceptable carrier (such as sterile, pyrogen-free physiological saline solution, or sterile, pyrogen-free phosphate-buffered saline solution). In general, the compositions are prepared by contacting and combining viral particles produced as above with a pharmaceutically acceptable carrier. The viral particles of the composition may be live, killed, fixed or lyophilized, as is most suitable for the intended use. The viral particles are included in the composition in an immunogenic amount, the amount to be determined by the intended use. The immunogenic activity of a given amount of the virus of the present invention may be determined by any of a number of methods known in the art. The increase in titer of antibody against a particular viral antigen upon administration may be used as a criteria for immunogenic activity.

Subjects which may be administered the live attenuated viruses and formulations disclosed herein include both human subjects and animal subjects (e.g., the veterinary treatment of primates such as owl monkeys, marmosets and chimpanzees, and other mammalian species such as dogs, cats, pigs, and horses, and non-mammalian species such as birds (chickens, turkeys, etc.)).

Pharmaceutical formulations of the present invention comprise an immunogenic amount of a live attenuated virus as disclosed herein in combination with a pharmaceutically acceptable carrier. An “immunogenic amount” is an amount of the attenuated virus sufficient to evoke an immune response in the subject to which the virus is administered. The particular dose employed is not critical, and depends upon the type and condition of the subject, the route of administration, etc.

Techniques to determine a particular immunogenic amount of the viral particles of the present invention will be apparent to those of ordinary skill in the art. For example, the active agent (viral particles or preparations thereof) may be given in an amount of from 0.01 to 100 μg per Kg body weight (e.g., 0.5 or 1.0 μg per Kg).

Administration of the live attenuated viruses disclosed herein may be carried out by any suitable means, including both parenteral injection (such as intraperitoneal, subcutaneous, or intramuscular injection), by oral administration, and by topical application of the virus (typically carried in the pharmaceutical formulation) to an airway surface. Topical application of the virus to an airway surface can be carried out by intranasal administration (e.g., by use of a dropper, swab, or inhaler which deposits a pharmaceutical formulation intranasally). Topical application of the virus to an airway surface can also be carried out by inhalation administration, such as by creating respirable particles of a pharmaceutical formulation (including both solid particles and liquid particles) containing the virus as an aerosol suspension, and then causing the subject to inhale the respirable particles. Methods and apparatus for administering respirable particles of pharmaceutical formulations are well known, and any conventional technique can be employed. See, e.g., U.S. Pat. No. 5,304,125 to D. Leith; U.S. Pat. No. 5,299,566 to C. Davis and R. Snyder; U.S. Pat. No. 5,290,550 to R. Fisher and W. Metzger; and U.S. Pat. No. 5,292,498 to R. Boucher.

Oral vaccine formulations may be made from a culture of cells producing live virus containing the desired attenuating mutations in accordance with known techniques. The culture itself may be administered to the subject; the culture may be optionally filtered and/or clarified; stabilizers such as sucrose, MgCl₂, etc. may be added to the media. Pharmaceutically acceptable carriers for oral administration may be a syrup, elixir, lozenge, etc. The vaccine formulation may be prepared in accordance with known techniques, such as illustrated by R. Purcell et al., Vaccine Against Hepatitis A Virus, U.S. Pat. No. 4,894,228.

While the viruses, methods and formulations of the present invention have been described above with reference to producing protective immunity by the administration of vaccine formulations, they may also be used to immunize animals to simply produce antibodies in animals, which antibodies may then be collected and used for the purpose of detecting and/or diagnosing various viral infections or the presence of viral particles in biological samples in accordance with conventional diagnostic techniques. See generally E. Maggio, Enzyme Immunoassay (1980); see also U.S. Pat. Nos. 4,659,678, 4,376,110, 4,275,149, 4,233,402, and 4,230,767.

5. Oligonucleotide Probes

An advantage of the instant invention is that it will permit detection of infection by wild-type virus even after vaccination has occurred. For example, a vaccine employing a whole or nearly whole virus will create an immune response to the virus that will preclude standard immunologic or nucleic acid detection of subsequent infection. The constructs of the instant invention, since they represent totally new creations at the level of the DNA, can easily be distinguished by molecular probing. Thus, probes can be made that will be specific for the wild type virus and that will not hybridize to a virus of the instant invention, and probes can be made that will specifically bind to the virus of the instant invention and not the wild type virus.

Thus, a further aspect of the present invention is an oligonucleotide probe useful for distinguishing between (i) an attenuated virus, not naturally occurring, containing at least 1 additional methylation site in the genome of the virus compared to the corresponding wild-type virus, and (ii) the corresponding wild-type virus, with the oligonucleotide probe selected from the group consisting of: (a) oligonucleotide probes that selectively hybridize to the nucleic acid of an attenuated virus of (i) above, and which do not hybridize to the nucleic acid of the wild-type virus of (ii) above under the same hybridization conditions; and (b) oligonucleotide probes that selectively hybridize to the nucleic acid of a wild-type virus of (ii) above, and which do not hybridize to the nucleic acid of the attenuated virus of (i) above under the same hybridization conditions. The probe may be of any suitable length so long as the desired specificity of binding is achieved. Such probes are typically at least 8 to 12 nucleotides in length and can be up to 20-40 nucleotides or more in length. The probe may be of any suitable nucleic acid, including DNA and RNA. The probe may be labeled with or conjugated to a detectable group (e.g., a radioisotope such as ³²P, ¹²⁵I, ¹³¹I, ³H, ¹⁴C, or ³⁵S; an enzyme such as horseradish peroxidase or alkaline phosphatase) by a variety of techniques, including direct covalent bond. The probe may be one probe or a member of a pair of probes useful for a nucleic acid amplification procedure, such as polymerase chain reaction (PCR), ligase chain reaction (LCR), or strand displacement amplification (SDA). Techniques for use of such probes are known to those skilled in the art. See, for example, U.S. Pat. No. 4,358,535 to Falkow and Mosley; U.S. Pat. No. 4,302,204 to Wahl and Stark; U.S. Pat. No. 4,994,373 to Stavrianopoulos; U.S. Pat. No. 5,270,184 to Walker et al.; and, for PCR, U.S. Pat. Nos. 4,683,195, 4,683,202, 4,800,159 and 4,965,188.

The present invention is explained in greater detail in the following non-limiting Examples.

### EXAMPLE 1

### Introduction of CpG Sites in HIV-1 Genome

The genomic sequence of HIV-1 strain HIVHXB2CG (see, e.g., F. Wong-Staal et al., Nature 313, 277-284 (1985) was obtained from GENBANK (Accession Number k03445). Sites in the sequence in which silent substitution mutations could be added to the genome to introduce additional CpG segments therein were identified and a new DNA encoding a non-natural derivative of the HIV-1 genome is synthesized as follows.

Single stranded DNA segments 75 bases in length are synthesized by phosphoramidate chemistry on an Applied Biosystems Model 394 DNA/RNA Synthesizer (Applied Biosystems Inc., 850 Lincoln Centre Drive. Foster City, Calif., 94404 USA). Each 75 base pair double-stranded DNA segment is deprotected at 55° C. for 12 hours and dried to remove ammonium hydroxide. The trityl group is left on at the deprotecting step. The full-length 75 base-pair segment is then separated from shorter “failure” segments in the preparation with NENSORB™ chromatography. This serves to avoid adding the shorter failure segments to the elongated segment.

Complementary segments are made and annealed together, with overlapping ends of 4 bases, to produce a double-stranded DNA segment 75 bases in length. Each new 75 base-pair double-stranded segment is sequentially ligated to the previous segment to build up an elongated double-stranded DNA segment that ultimately becomes the entire modified HIV-1 genome (HIV-1^{CpG-1}), given in SEQ ID NO: 1.

Appropriate splice segments are added to each end of the complete genome by conventional techniques and the genome inserted into an expression vector.

### COMPARATIVE EXAMPLE A

### Comparison of CpG Sites in HIV-1 Strain HIVHXB2CG and Strain HIV-1^{CpG1}

The CpG content of the HIVHXB2CG genome is illustrated in graph form in FIG. 2. The gene structure of HIV is incorporated into this graph for clarity.

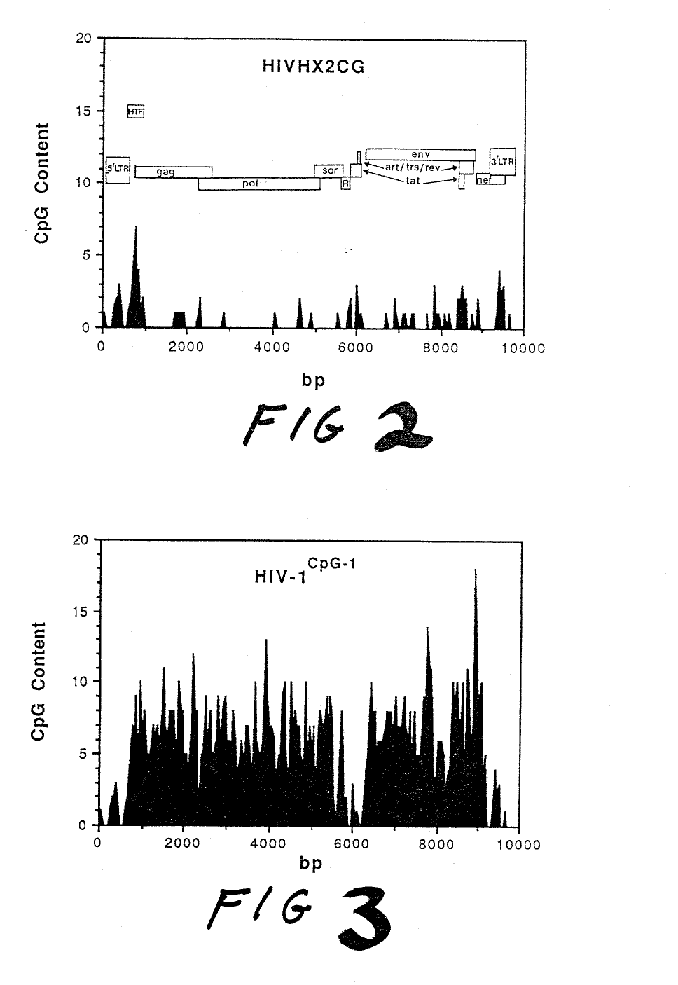

The CpG content of the HIV-1^{CpG1} genome is illustrated by graph in FIG. 3. Note the dramatic increase in CpG content as compared to the wild-type genome shown in FIG. 2. HIV-1^{CpG1} has 948 new CpG sites as compared to HIVHXB2CG (representing a more than tenfold increase in CpG segments: 97 in HIVHXB2CG; 1045 in HIV-1^{CpG1}). The ratio of expected over observed CpG dinucleotides (CpG^{o/e} in HIV-1^{CpG-1} is increased from a value of 0.22 in HIVHXB2CG to a value of 1.68 in HIV-1^{CpG-1}. This represents an approximately 8-fold increase in CpG^{o/e}. In extreme cases(e.g. those in which many hundreds of new CpG methylation sites have been inserted into the viral genome, as in the example modified genome, HIV-1^{CpG-1}) this will result in an increase in the GC/AT ratio above that observed in the wild type virus. Thus, the GC/AT ratio in HIB-1^{CpG-1}, is equal to 1.05 as compared to 0.74 in the wild type genome, HIVHXB2CG. The base count in HIV-1C^{CpG-1}, as compared to HTVRXB2CG is as follows;

| | HIVHXB2CG | HIV-1^{CpG-1} |
|---|---|---|
| Adenines | 3411 | 2796 |
| Cytosines | 1773 | 2197 |
| Guanines | 2370 | 2772 |
| Thymines | 2164 | 1953 |

This represents a loss of 615 adenines and 211 thymines in HIV-1^{CpG-1}, as compared to HIVHXB2CG and a gain of 424 cytosines and 402 guanines in HIV-1^{CpG-1} as compared to HTVIXB2CG. The ration of GC/AT will not be increased significantly in those modified genomes in which only a small number of CpGs need to be inserted (e.g.<10) to interrupt the viral life cycle. The GC/AT ratio in HIVHXB2CG is 0.74; while the GC/AT ratio in HIV-1^{CpG-1}, is 1.05.

### EXAMPLE 2

### Expression of HIV-1 Genome in Insect Cells

The BACKPACK˜ baculovirus expression system is obtained from Clontech Inc. (Telephone Number in USA. 415424-8222). The genomic DNA segment described in Example 1 above is ligated into the multiple cloning site of pBacPAK8˜ (or PBacPAK9˜) to produce a recombinant vector, with expression of the genomic DNA driven by the strong AcHNPV polyhedrin promoter in the vector. Cultured Spodoptera frugiperda cells are transformed with the recombinant vector and the virus of the invention is produced in the cultured cells in accordance with the manufacturer's instructions.

The foregoing examples are illustrative of the present invention, and are not to be construed as limiting thereof. The invention is defined by the following claims, with equivalents of the claims to be included therein.

That which is claimed is:

1. An attenuated virus, not naturally occurring, containing at least 1 additional methylation site in the genome of said virus compared to the corresponding wild-type virus.

2. An attenuated virus according to claim 1, said virus comprising a viral capsid containing said genome.

3. An attenuated virus of claim 1, containing at least 10 additional methylation sites over the corresponding wild-type virus.

4. An attenuated virus of claim 1, containing at least 100 additional methylation sites over the corresponding wild-type virus.

5. An attenuated virus of claim 1 wherein said methylation site is a CG segment.

6. An attenuated virus according to claim 1, wherein said virus is a DNA virus.

7. An attenuated virus according to claim 1, wherein said virus is a retrovirus.

8. An attenuated virus of claim 1 wherein said virus is a retrovirus selected from the group consisting of B-type retroviruses, C-type retroviruses, D-type retroviruses, Lentiviruses, T-cell leukemia viruses, and foamy viruses.

9. An attenuated virus of claim 1, wherein said virus is HIV-1.

10. An attenuated virus of claim 1, wherein said virus is SIV.

11. An attenuated virus of claim 1, wherein said virus is HTLV-1.

12. An attenuated virus of claim 1, wherein said virus is a retrovirus and wherein an attenuating deletion mutation is included therein.

13. A DNA encoding a virus of claim 1.

14. An expression vector containing a DNA of claim 13.

15. An expression vector of claim 14, wherein said expression vector is a Baculovirus.

16. A host cell containing a DNA of claim 13 and capable of expressing the encoded virus, which host cell does not methylate said DNA sufficient to inactivate the expression of the encoded viral genome.

17. A host cell according to claim 16, which host cell lacks capacity to methylate DNA because of treatment of said host cell with a methylation inhibitor.

18. A host cell according to claim 17 wherein said methylation inhibitor is 5-azadeoxycytidine or 5-azacytidine.

19. A pharmaceutical formulation comprising a virus according to claim 1 in combination with a pharmaceutically acceptable carrier.

20. A formulation according to claim 19, wherein said formulation is an oral formulation.

21. A formulation according to claim 19, wherein said formulation is a parenterally injectable vaccine formulation.

22. A formulation according to claim 19, wherein said formulation is an inhalation formulation.

23. A method of producing an immune response in a subject, comprised of administering a virus of claim 1 to said subject in an amount effective to produce an immune response in said subject.

24. A method according to claim 23, wherein said administering step is carried out by orally administering said virus to said subject.

25. A method according to claim 23, wherein said administering step is carried out by parenterally injecting said virus into said subject.

26. A method according to claim 23, wherein said subject is an animal subject.

27. A method according to claim 23, wherein said subject is a human subject.

28. A method of making an attenuated virus, not naturally occurring, containing at least 1 additional methylation site in the genome of said virus compared to the corresponding wild-type virus; said method comprising:
providing a host cell containing an expression vector, said expression vector containing a DNA encoding said attenuated virus, which host cell does not methylate said DNA sufficient to inactivate the expression of the encoded viral genome; and
expressing said attenuated virus in said host cell.

29. A method according to claim 28, the genome of said virus containing at least 10 additional methylation sites over the corresponding wild-type virus.

30. A method according to claim 28, wherein said virus is a DNA virus.

31. A method according to claim 28, wherein said virus is a retrovirus.

32. A method according to claim 28, wherein said expression vector is a Baculovirus.

33. A method according to claim 28, wherein said host cell is an insect cell.

34. A method according to claim 28, wherein said host cell is a mammalian cell.

35. An oligonucleotide probe useful for distinguishing between (i) an attenuated virus, not naturally occurring, containing at least 1 additional methylation site in the genome of said virus compared to the corresponding wild-type virus, and (ii) said corresponding wild-type virus, said oligonucleotide probe selected from the group consisting of:
(a) oligonucleotide probes that selectively hybridize to the nucleic acid of an attenuated virus of (i) above, and which do not hybridize to the nucleic acid of the wild-type virus of (ii) above under the same hybridization conditions; and
(b) oligonucleotide probes that selectively hybridize to the nucleic acid of a wild-type virus of (ii) above, and which do not hybridize to the nucleic acid of the attenuated virus of (i) above under the same hybridization conditions.

36. An oligonucleotide probe according to claim 35 conjugated to a detectable group.

37. An oligonucleotide probe according to claim 35, wherein said probe is a PCR extension primer.


SEQUENCE LISTING (1 sequence)

SEQ ID NO: 1 - DNA (cDNA), 9718 bp; single-stranded, linear
tggaagggctaattcactcccaacgaagacaagatatccttgatctgtggatctaccacacacaaggctacttccctgattagcagaactacacaccagggccagggatcagatatccactgacctttggatggtgctacaagctagtaccagttgagccagagaagttagaagaagccaacaaaggagagaacaccagcttgttacaccctgtgagcctgcatggaatggatgacccggagagagaagtgttagagtggaggtttgacagccgcctagcatttcatcacatggcccgagagctgcatccggagtacttcaagaactgctgacatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcccgcacggcaagaggcgaggggcggcgactggtgagtacgccaaaaattttgactagcggaggctagaaggagagagatgggcgcgcgcgcgtcggtattatcgggcggcgaattagatcgatgggaaaaaattcggttacggccgggcggaaagaaaaaatataaattaaaacatatcgtatgggcgtcgcgcgagctcgaacgattcgcggttaatccgggcctgttagaaacgtcggaaggctgtcgacaaatactcggacagctacaaccgtcgcttcagacgggatcggaagaacttcgatcgttatataatacggtcgcgacgctctattgcgtccatcaacggatcgagataaaagacacgaaggaagcgttagacaagatcgaggaagagcaaaacaaatcgaagaaaaaagcgcagcaagcggcggcggacacgggacactcgaatcaggtctcgcaaaattacccgatcgtgcagaacatccaggggcaaatggtacatcaggcgatatcgccgcgaacgttaaacgcgtgggtaaaagtcgtcgaagagaaggcgttctcgccggaagtgataccgatgttttcggcgttatcggaaggagcgacgccgcaagatttaaacacgatgctaaacacggtcggcggacatcaagcggcgatgcaaatgttaaaagagacgatcaacgaggaagcggcggaatgggatcgcgtgcatccggtgcacgcggggccgatcgcgccgggccagatgcgcgaaccgcgcggatcggacatcgcgggaacgacgtcgacgcttcaggaacaaatcggatggatgacgaataatccgccgatcccggtcggcgaaatttataaacgatggataatcctcggattaaataaaatcgtacgaatgtattcgccgacgtcgattctcgacatacgacaaggaccgaaggaaccgtttcgcgactacgtcgaccggttctataaaacgctacgcgcggagcaagcgtcgcaggaggtaaaaaattggatgacggaaacgttgttggtccaaaacgcgaacccggattgtaagacgattttaaaagcgttgggaccggcggcgacgctcgaagaaatgatgacggcgtgtcagggcgtcggcggaccgggccataaggcgcgcgttttggcggaagcgatgtcgcaagtaacgaattcggcgacgataatgatgcagcgcggcaattttcggaaccaacgaaagatcgttaagtgtttcaattgcggcaaagaagggcacacggcgcgaaattgccgcgcgccgcggaaaaagggctgttggaaatgcggaaaggaaggacaccaaatgaaagattgtacggagcgacaggcgaattttctcgggaagatctggccgtcgtacaagggacggccggggaattttcttcagtcgcgaccggagccgacggcgccgccggaagagtcgttccggtcgggcgtcgagacgacgacgccgccgcagaagcaggagccgatagacaaggaactgtatccgttaacgtcgctccggtcgctcttcggcaacgacccgtcgtcgcaataaagataggggggcaactaaaggaagctctattagatacaggagcagatgatacagtattagaagaaatgtcgttgccgggacgatggaaaccgaaaatgatcggcggaatcggcggttttatcaaagtacgacagtacgatcagatactcatcgaaatctgcggacataaagcgatcggtaccgtactcgtcggaccgacgccggtcaacataatcggacgaaatctgttgacgcagatcggttgcacgttaaattttccgatttcgccgatcgagacggtaccggtaaaattaaagccgggaatggacggcccgaaagttaaacaatggccgttgaccgaagaaaaaataaaagcgctcgtcgaaatttgtacggagatggaaaaggaagggaaaatttcgaaaatcgggccggaaaatccgtacaatacgccggtattcgcgataaagaaaaaagactcgacgaaatggcgaaaactcgtcgatttccgcgaacttaataagcgaacgcaagacttctgggaagttcaactcggaataccgcatccggcggggttaaaaaagaaaaaatcggtaacggtactcgatgtcggcgacgcgtatttttcggttccgctcgacgaagacttccggaagtatacggcgtttacgataccgtcgataaacaacgagacgccggggattcgatatcagtacaacgtgcttccgcagggatggaaaggatcgccggcgatattccaatcgtcgatgacgaaaatcctcgagccgtttcgaaaacaaaatccggacatcgttatctatcaatacatggacgatttgtacgtcggatcggacctcgaaatcgggcagcatcgaacgaaaatcgaggagctgcgacaacatctgttgcggtggggacttacgacgccggacaaaaaacatcagaaagaaccgccgttcctttggatgggttacgaactccatccggataaatggacggtacagccgatcgtgctgccggaaaaagactcgtggacggtcaacgacatacagaagctcgtcgggaaattgaattgggcgtcgcagatttacccggggattaaagtacggcaattatgtaaactccttcgcggaacgaaagcgctaacggaagtaataccgctaacggaagaagcggagctcgaactcgcggaaaaccgcgagattctaaaagaaccggtacacggcgtgtattacgacccgtcgaaagacttaatcgcggaaatacagaagcaggggcaaggccaatggacgtatcaaatttatcaagagccgtttaaaaatctgaaaacgggaaaatacgcgcgcatgcggggcgcgcacacgaacgacgtaaaacaattaacggaggcggtgcaaaaaataacgacggaatcgatcgtaatatggggaaagacgccgaaatttaaactgccgatacaaaaggaaacgtgggaaacgtggtggacggagtattggcaagcgacgtggattccggagtgggagttcgttaatacgccgccgctcgtgaaattatggtaccagctcgagaaagaaccgatcgtcggcgcggaaacgttctacgtcgacggcgcggcgaaccgcgagacgaaactcggaaaagcgggatacgttacgaatcgcggacgccaaaaagtcgtcacgctaacggacacgacgaatcagaagacggagttacaagcgatttatctcgcgttgcaggattcgggactcgaagtaaacatcgtaacggactcgcaatacgcgttaggaatcattcaagcgcaaccggatcaatcggaatcggagttagtcaatcaaataatcgagcagttaataaaaaaggaaaaggtctatctcgcgtgggtaccggcgcacaaaggaatcggcggaaacgaacaagtcgataaattagtctcggcgggaatccggaaagtactatttttagacggaatcgataaggcgcaagacgaacacgagaaatatcactcgaattggcgcgcgatggcgtcggattttaacctgccgccggtcgtcgcgaaagaaatcgtcgcgtcgtgcgataaatgtcagctaaaaggcgaagcgatgcacggacaagtcgactgttcgccgggaatatggcaactcgattgtacgcatttagaaggaaaagttatcctcgtcgcggttcacgtcgcgtcgggatatatcgaagcggaagttattccggcggaaacggggcaggaaacggcgtattttcttttaaaattagcgggacgatggcccgtaaaaacgatacatacggacaatggctcgaatttcaccggcgcgacggttcgcgcggcgtgttggtgggcgggaatcaagcaggaattcggaattccgtacaatccgcaatcgcaaggcgtcgtcgaatcgatgaataaagaattaaagaaaattatcggacaggtacgcgatcaggcggaacatcttaagacggcggtacaaatggcggtattcatccacaattttaaacgaaaaggcgggattggcgggtactcggcgggcgaacgaatcgtcgacataatcgcgacggacatacaaacgaaagaattacaaaaacaaattacgaaaattcaaaattttcgcgtttattaccgcgactcgcgaaattcgctttggaaaggaccggcgaagctcctctggaaaggcgaaggcgcggtcgtaatacaagataattcggacataaaagtcgtgccgcgacgaaaagcgaagatcattcgcgattatggaaaacagatggcaggtgatgattgtgtggcaagtagacaggatgaggattcgcacgtggaaatcgctcgtaaaacaccatatgtacgtttcggggaaagcgcgcggatggttttatcgccatcactacgaatcgccgcatccgcgcatatcgtcggaagtacacatcccgctcggggatgcgcgcctcgtaataacgacgtattggggtctgcatacgggcgaacgcgactggcatctcggtcagggcgtctcgatcgaatggcgcaaaaagcgctattcgacgcaagtcgacccggaactcgcggaccaactaattcatctgtattacttcgactgtttttcggactcggcgatacgcaaggcgttactcggacacatcgtttcgccgcgctgcgaatatcaagcgggacataacaaggtcggatcgctacaatacctcgcgctcgcggcgttaataacgccgaaaaagataaagccgccgttgccgtcggttacgaaactgacggaggatcgatggaacaagccccagaagaccaagggccacagagggagccacacaatgaatggacactagagcttttagaggagcttaagaacgaagcggttcgccattttccgcgcatttggctccacggcttagggcaacatatctacgaaacgtatggggatacgtgggcgggcgtcgaagcgataataagaattctgcaacaactgctgtttatccattttcagaattgggtgtcgacgtagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcagtaagtagtacatgtaacgcaacctataccaatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgcgagtgaaggagaaatatcagcacttgtggcgatgggggtggcgatggggcacgatgctcctcgggatgttgatgatctgttcggctacggaaaaattgtgggtcacggtctattacggcgtaccggtgtggaaggaagcgacgacgacgctattttgcgcgtcggacgcgaaagcgtacgatacggaggtacataacgtttgggcgacgcatgcgtgcgtaccgacggacccgaacccgcaagaagtcgtactcgtaaacgtgacggaaaatttcgacatgtggaaaaacgacatggtcgaacagatgcatgaggatataatctcgttatgggatcaatcgctaaagccgtgcgtaaaattaacgccgctctgcgtttcgttaaagtgcacggatttgaagaatgatacgaatacgaattcgtcgtcggggcgaatgataatggagaaaggcgagataaaaaactgctcgttcaatatctcgacgtcgatacgcggtaaggtgcagaaagaatacgcgtttttttataaactcgatataataccgatcgataacgatacgacgtcgtattcgttgacgtcgtgtaacacgtcggtcattacgcaggcgtgtccgaaggtatcgttcgagccgattccgatacattattgtgcgccggcgggtttcgcgattctaaaatgtaataataagacgttcaacggaacgggaccgtgtacgaacgtctcgacggtacaatgtacgcacggaattcggccggtcgtatcgacgcaactgctgttaaacggctcgctcgcggaagaagaggtcgtaattcgatcggtcaatttcacggacaacgcgaaaacgataatcgtacagctgaacacgtcggtcgaaattaattgtacgcgaccgaacaacaatacgcgaaaacgaatccgtatccagcgcggaccggggcgcgcattcgttacgatcggaaaaatcggaaatatgcgacaagcgcattgtaacatttcgcgcgcgaaatggaataacacgttaaaacagatcgattcgaaattacgcgaacaattcggaaataataaaacgataatctttaagcaatcgtcgggcggcgacccggaaatcgtaacgcactcgtttaattgtggcggcgaatttttctactgtaattcgacgcaactgtttaattcgacgtggtttaattcgacgtggtcgacggaagggtcgaataacacggaaggatcggacacgatcacgctcccgtgccgaataaaacaaattataaacatgtggcagaaagtcggaaaagcgatgtacgcgccgccgatctcgggacaaattcgatgttcgtcgaatattacggggctgctattaacgcgcgacggcggtaattcgaacaacgagtccgagatcttccgactcggcggcggcgatatgcgcgacaattggcgatcggaattatataaatataaagtcgtaaaaatcgaaccgctcggcgtcgcgccgacgaaggcgaagcgacgcgtcgtgcagcgcgaaaaacgcgcggtcggaatcggcgcgttgttcctcgggttcctcggcgcggccggatcgacgatgggcgcggcgtcgatgacgctgacggtacaggcgcgacaattattgtcgggtatcgtgcagcagcagaacaatttgctgcgcgctatcgaggcgcaacagcatctgttgcaactcacggtctggggcatcaagcagctccaagcgcgaatcctcgcggtcgaacgatacctaaaggatcaacagctcctcgggatttggggttgctcgggaaaactcatttgcacgacggcggtgccgtggaatgcgtcgtggtcgaataaatcgctcgaacagatctggaatcacacgacgtggatggagtgggaccgcgaaattaacaattacacgtcgttaatacactcgttaattgaagaatcgcaaaaccagcaagaaaagaatgaacaagaattactcgaactcgataaatgggcgtcgttgtggaattggtttaacataacgaattggctgtggtatataaaattattcataatgatcgtcggcggcctcgtcggtttacgaatcgttttcgcggtactttcgatcgtgaatcgcgttcggcagggatattcgccgttatcgtttcagacccacctcccaatcccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataagatgggcggcaagtggtcgaaatcgtcggtgattggatggcttacggtacgcgaacgcatgcgccgcgccgagccggcggcggacggcgtcggcgccgcgtcgcgcgacctggaaaaacacggcgcgatcacgtcgtcgaacacggcggcgacgaacgcggcgtgcgcgtggctcgaagcgcaagaggaggaggaggtcggttttccggtcacgccgcaggtaccgttacgcccgatgacgtacaaggcggcggtcgatctttcgcactttttaaaagaaaagggcggactcgaagggctaattcactcgcaacgccgccaagatatcctcgatctgtggatctaccacacgcaaggctacttcccggattgacagaactacacaccagggccaggggtcagatatccactgacctttggatggtgctacaagctagtaccagttgagccagataagatagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca